(19) United States
(12) Patent Application Publication
(10) Pub. No.: US 2026/0334874 A1
(43) Pub. Date: Oct. 29, 2026

(54) CIRCADIAN RHYTHM ADAPTATION

(71) Applicant: Polar Electro Oy, Kempele (FI)

(72) Inventors: Riikka Ahola, Kempele (FI); Topi Korhonen, Kempele (FI); Matti Luomala, Kempele (FI); Kaisu Martinmäki, Kempele (FI); Lotta Rönnberg, Kempele (FI); Eve Vallström, Kempele (FI)

(73) Assignee: Polar Electro Oy, Kempele (FI)

(21) Appl. No.: 19/124,266

(22) PCT Filed: Nov. 2, 2023
(86) PCT No.: PCT/FI2023/050610

§ 371 Date: Apr. 24, 2025

(30) Foreign Application Priority Data
Nov. 4, 2022 (GB) 2216413.1

(51) Int. Cl. A61B 5/00; A61B 5/11

(52) U.S. Cl. CPC A61B 5/4857; A61B 5/1112; A61B 5/681

(57) ABSTRACT

A method for estimating adaptation of a circadian rhythm of a user, which includes: measuring the user's circadian phase by using at least one of a skin temperature sensor, a heart activity sensor, and a motion sensor; measuring the user's sleep-wake rhythm by using at least one of the heart activity sensor and the motion sensor, and detecting a change in the user's sleep-wake rhythm on the basis of the measurements; determining a setpoint phase for the circadian phase on the basis of the changed sleep-wake rhythm; determining a target circadian phase based on the user's location; determining a circadian adaptation rate based on a first adaptation rate, if the setpoint phase and the target phase are in same direction with respect to the current circadian phase, and determining the circadian adaptation rate based on a second adaptation rate, if the setpoint phase and the target phase are to opposite directions from the current circadian phase, wherein the second adaptation rate is smaller than the first adaptation rate; and outputting the determined circadian adaptation rate or a parameter derivative of the computed circadian adaptation rate via an interface to be presented to the user.

### CROSS-REFERENCE TO RELATED APPLICATIONS

This application is a national phase application of International Application No. PCT/FI2023/050610, filed Nov. 2, 2023, which claims benefit and priority to Great Britain Application No. 2216413.1, filed Nov. 4, 2022, which are incorporated by reference herein in their entireties.

### BACKGROUND

This invention relates to estimating adaptation of a circadian rhythm of a user.

### SUMMARY

A circadian rhythm (equivalently circadian process, circadian cycle) may be understood as a human internal physiological process regulating a natural sleep-wake rhythm of a person. The circadian rhythm repeats approximately every 24 hours. The circadian cycle comprises a circadian phase that can be understood as reflecting timing of different states of the circadian rhythm during a (calendar) day. Different persons have different natural circadian phases, and many everyday factors influence the person's capability and willingness to follow the natural circadian phase.

The circadian rhythm is a function of a person's sleeping rhythm. A sleep-wake rhythm provides a setpoint for the circadian rhythm. The person may need to change the sleep-wake rhythm in various situations such as travelling across time zones, change to summer/wintertime, shift work, social activities, and other changes in sleeping rhythm and/or in environments. The change causes circadian misalignment between the circadian rhythm and the sleep-wake rhythm which reduces alertness even for several days. The term circadian misalignment refers to a phase shift between one's internal circadian rhythm vs. actual sleep-wake rhythm. When one travels across more than two time zones quickly, one's internal clock needs time to adjust to the new daylight-nighttime schedule at one's destination. After arriving, one's circadian rhythm is out of sync with a sleep-wake rhythm of the new destination, but it will adjust gradually to the new time zone. It is generally agreed that flying eastward may cause more severe jet lag symptoms than flying westward. This is because one's body can adapt more quickly to staying up late than going to bed earlier than normal. Twice a year, there is a transition into and out of daylight-saving time in several countries. In consequence people experience a mini jet lag as their internal circadian rhythms and sleep-wake rhythms as well as environmental light-dark rhythms fall out of synch. Many people engage in rotating shift work, and they experience a mini jet lag on a weekly basis. In addition, even more people voluntarily shift their bedtimes and wake-up times between weekdays and weekends leading to recurrent circadian misalignment, called social jetlag.

U.S. Pat. No. 10,448,829 discloses a method for detecting, estimating, and dis-playing a disturbance to a biological rhythm of a user, wherein the disturbance is caused by travelling, irregular sleeping habits, or shift work. It also estimates a recovery time from the phase shift. While it recognizes the sunlight as a factor affecting the circadian rhythm, it fails to take it into account as a factor in connection with recovering from the disturbance, for example.

According to an aspect, there is a method for estimating adaptation of a circadian rhythm of a user, the method comprising: measuring the user's circadian phase by using at least one of a skin temperature sensor, a heart activity sensor, and a motion sensor, measuring the user's sleep-wake rhythm by using at least one of the heart activity sensor and the motion sensor, and detecting a change in the user's sleep-wake rhythm on the basis of the measurements, determining a setpoint phase for the circadian phase on the basis of the changed sleep-wake rhythm, determining a target circadian phase based on the user's location, determining a circadian adaptation rate based on a first adaptation rate, if the setpoint phase and the target phase are in same direction with respect to the current circadian phase, and determining the circadian adaptation rate based on a second adaptation rate, if the setpoint phase and the target phase are to opposite directions from the current circadian phase, wherein the second adaptation rate is smaller than the first adaptation rate; and outputting the determined circadian adaptation rate or a parameter derivative of the determined circadian adaptation rate via an interface to be presented to the user.

According to an aspect, there is an apparatus for estimating adaptation of a circadian rhythm of a user, the apparatus comprising measuring the user's circadian phase by using at least one of a skin temperature sensor, a heart activity sensor, and a motion sensor, measuring the user's sleep-wake rhythm by using at least one of the heart activity sensor and the motion sensor, and detecting a change in the user's sleep-wake rhythm on the basis of the measurements, determining a setpoint phase for the circadian phase on the basis of the changed sleep-wake rhythm, determining a target circadian phase based on the user's location, determining a circadian adaptation rate based on a first adaptation rate, if the setpoint phase and the target phase are in same direction with respect to the current circadian phase, and determining the circadian adaptation rate based on a second adaptation rate, if the setpoint phase and the target phase are to opposite directions from the current circadian phase, wherein the second adaptation rate is smaller than the first adaptation rate; and outputting the determined circadian adaptation rate or a parameter derivative of the determined circadian adaptation rate via an interface to be presented to the user.

According to an aspect, there is a computer program product embodied on a distribution medium readable by a computer and comprising instructions, which, when loaded into an apparatus, execute: measuring the user's circadian phase by using at least one of a skin temperature sensor, a heart activity sensor, and a motion sensor, measuring the user's sleep-wake rhythm by using at least one of the heart activity sensor and the motion sensor, and detecting a change in the user's sleep-wake rhythm on the basis of the measurements, determining a setpoint phase for the circadian phase on the basis of the changed sleep-wake rhythm, determining a target circadian phase based on the user's location, determining a circadian adaptation rate based on a first adaptation rate, if the setpoint phase and the target phase are in same direction with respect to the current circadian phase, and determining the circadian adaptation rate based on a second adaptation rate, if the setpoint phase and the target phase are to opposite directions from the current circadian phase, wherein the second adaptation rate is smaller than the first adaptation rate; and outputting the determined circadian adaptation rate or a parameter derivative of the determined circadian adaptation rate via an interface to be presented to the user.

### BRIEF DESCRIPTION OF THE DRAWINGS

In the following the invention will be described in greater detail by means of preferred embodiments with reference to the attached drawings, in which

FIG. 1 illustrates an example system to which the embodiments of the invention may be applied;

FIG. 2 illustrates an embodiment of the circadian rhythm detection method;

FIG. 3 illustrates an embodiment of the circadian rhythm detection method wherein such skin temperature measurements data for which a corresponding skin contact measurement data indicates no contact to skin is excluded from fitting to the cosine waveform;

FIG. 4 illustrates an embodiment of the circadian rhythm detection method wherein the amount of skin temperature measurement is checked against a first threshold;

FIG. 5 illustrates varying the phase of the cosine waveform;

FIG. 6 illustrates an embodiment of the circadian rhythm detection method, wherein the circadian phase is chosen between first phase value and a combination of the first phase value and the second phase value;

FIG. 7 illustrates an embodiment of determining the circadian phase via sleep-wake rhythm;

FIG. 8 illustrates an embodiment of an apparatus for detecting the circadian rhythm and for adaptation of the circadian rhythm;

FIG. 9 illustrates an embodiment of the method for adaptation of circadian rhythm;

FIG. 10 illustrates an embodiment of the method for adaptation of circadian rhythm, wherein the circadian adaption rate is determined with the user's light exposure;

FIG. 11 illustrates an embodiment of the method for adaptation of circadian rhythm, wherein the circadian adaption rate is determined depending on the westward or eastward setpoint phase; and

FIG. 12 illustrates an embodiment of an iterative algorithm for measuring the circadian phase.

### DETAILED DESCRIPTION

The following embodiments are exemplifying. Although the specification may refer to “an”, “one”, or “some” embodiment(s) in several locations of the text, this does not necessarily mean that each reference is made to the same embodiment(s), or that a particular feature only applies to a single embodiment. Single features of different embodiments may also be combined to provide other embodiments.

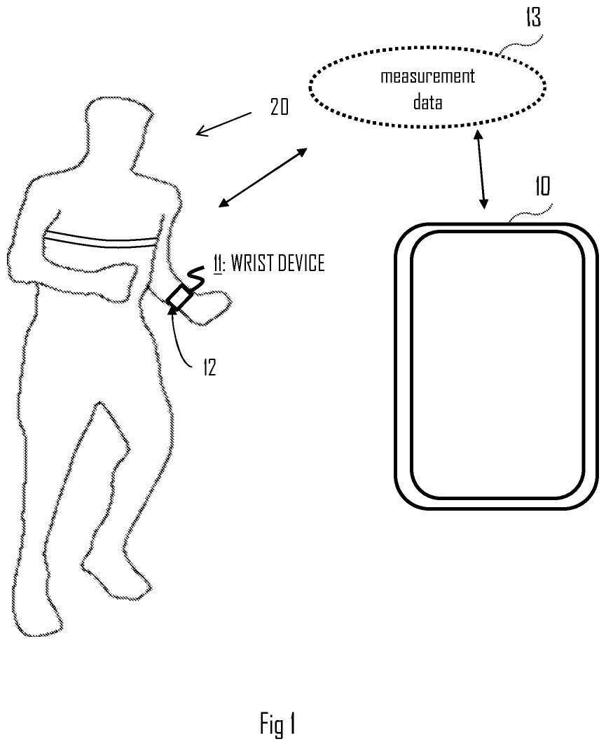

FIG. 1 illustrates a measurement system comprising a sensor device that may be used in the context of some embodiments of the present invention. The user 20 may wear a wearable device, such as a wrist device 11. The wrist device 11 may be, for example, a smart watch, a smart device, sports watch, and/or an activity tracking apparatus (e.g. bracelet, arm band, wrist band, mobile phone, glasses). In an embodiment, the wrist device 11 is an activity tracking apparatus or a wearable activity tracking apparatus. This may mean that said apparatus may be worn in the wrist or in other parts of the user 20, such as but not limited to forearm, bicep area, neck, forehead, and/or leg. The measurement system may further comprise a strap or a band for attaching the wearable device to the user, e.g. to a wrist. Data retrieved from wrist device 11 may also be used determine sleep start time and sleep end time, and skin temperature of the user 20. The embodiments described herein use wrist skin temperature and sleep start and wake-up times to determine circadian rhythm characteristics. Body core temperature represents the circadian rhythm in great detail, as known in the literature, and it peaks at daytime and is lowest at night. Therefore, it is possible to measure the circadian rhythm by measuring the body core temperature. The wrist skin temperature shows opposite patterns, being lowest at daytime and highest at night. However, they both follow the same rhythm and, therefore, it is possible to measure the circadian rhythm also from the skin temperature. A skin temperature sensor may be implemented in the wearable device as one of sensors 12 of the wearable device.

In the example illustrated in FIG. 1, the measurement system comprises at least a processing circuitry configured to analyse measurement data 13 measured from the user 20, for example by carrying out methods described in more detail below. The processing circuitry may be realised in the wearable device worn by the user, or the processing circuitry may be realised in a user device 10 such as a smart phone or a tablet computer, or the processing circuitry may be realised in a server computer such as a cloud server. The measurement data 13 may be provided by at least one sensor device 12 which may be comprised in the wearable device 11, or the sensor device 12 may be external to the wearable device 11 but provided with data transfer capability with the wearable device 11. The wearable device 11, the user device 102, the server computer, and/or the sensor device 12 may be connectable over one or more networks, over a short-range wireless connection such as Bluetooth, or over a Universal Serial Bus (USB) connection.

The sensors of the sensor device 12 may employ one or more measurement technologies for measuring the activity and skin temperature of user 20. The activity measurements may be based on using a heart activity sensor At least one sensor device 12 may be configured to measure the skin temperature, and also the skin contact using optical or bioimpedance method so that those temperature samples, which are measured when sensor is not worn, can be detected and optionally excluded from the estimation of the circadian rhythm. The sensor device 12 may measure one or more of the following features from the user:motion, electrocardiogram (ECG), photoplethysmogram (PPG), bioimpedance, galvanic skin response, body temperature. For measuring the motion, the sensor device 12 may comprise an inertial sensor such as an accelerometer and/or gyroscope, or a magnetometer, or any a sensor fusion that is any combination of these motion sensors, and the sensor device 12 may output motion measurement data. The sensor device 12 measuring ECG or PPG may output heart activity measurement data 13. The sensor device 12 may comprise one or more electrodes attachable to the user's 20 skin to measure an electric property from the skin which, through appropriate signal processing techniques, may be processed into an ECG signal, bioimpedance signal, or galvanic skin response signal. In some techniques, the heart activity measurement data 13 may represent appearance of R waves of electric heart impulses. In PPG measurements a light emitted by a light emitter diode or a similar light source and reflected back from the user's 20 skin is be sensed by using a photo diode or a similar light sensing component. The sensed light is then converted into an electric measurement signal in the light sensing component and signal processing is used to detect desired signal components from the electric measurement signal. In the PPG measurements, P waves may be detected which enables computation of a PP interval and a heart rate, for example.

The user device 10 refers to a computing device (equipment, apparatus) and it may also be referred to as a user terminal, a user apparatus, mobile device, or a mobile terminal. Portable computing devices (apparatuses) include wireless mobile communication devices operating with or without a subscriber identification module (SIM) in hardware or in software, including, but not limited to, the following types of devices: mobile phone, smartphone, personal digital assistant (PDA), handset, laptop and/or touch screen computer, tablet (tablet computer), multimedia device, wearable computer, such as smart watch, and other types of wearable devices, such as clothing and accessories incorporating computer and advanced electronic technologies. The user device 102 may comprise one or more user interfaces. The one or more user interfaces may be any kind of a user interface, for example a screen, a keypad, a loudspeaker, a microphone, a touch user interface, an integrated display device, and/or external display device.

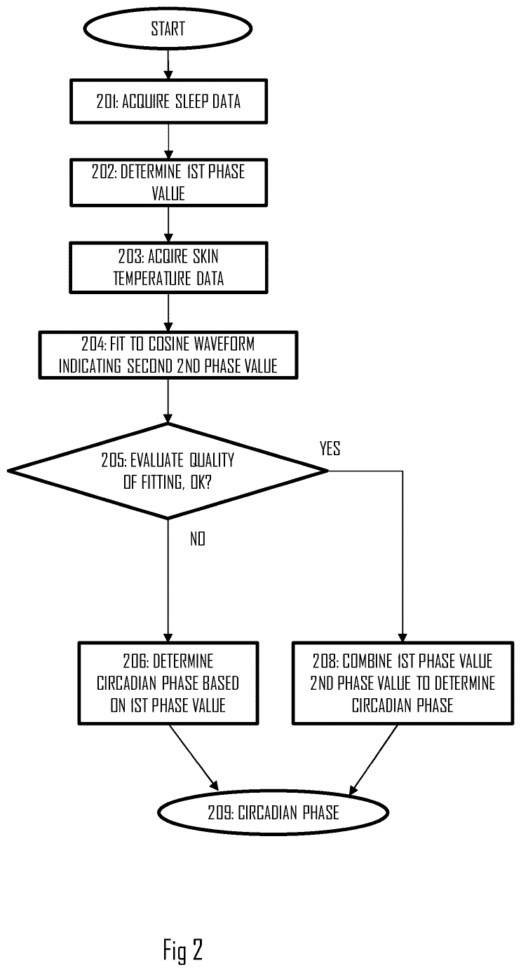

FIG. 2 is illustrating an embodiment, where there is a method for detecting a circadian rhythm of a user 20, wherein the method comprises acquiring 201 measurement data indicating the sleep time and determining a sleep-wake rhythm of the user 20 on the basis of the measurement data, and further determining 202 a first phase value based on the sleep-wake rhythm, and further acquiring 203 skin temperature measurement data for at least one 24-hour measurement period, and further fitting 204 the skin temperature measurement data to a cosine waveform, and performing evaluation 205 of a quality of the fitting and, if the quality is above a threshold, determining 207 a second phase value on the basis of the fitting, and if the quality of the fitting is above the threshold, determining 208 the user's 20 circadian phase as a combination of the first phase value and the second phase value, and if the quality of the fitting is below the threshold, determining 206 the user's 20 circadian phase based on the first phase value without the second phase value and outputting 209 the computed circadian phase or a parameter derivative of the computed circadian phase via an interface to be presented to the user 20. The second phase value may be determined before evaluation 205 of the fitting 204 or right after the fitting 204.

The measurement data acquired in block 201 may be acquired by using at least one of a heart activity sensor and a motion sensor of the sensor devices 12. The detection of the sleep start time and the sleep end time may be carried out according to the state of the art. The detection of the sleep start time and the sleep end time by using the heart activity sensor and/or the motion sensor is as such generally well known and there exist several commercially available products for performing this, e.g. Polar Vantage series by Polar Electro.

The embodiment of FIG. 2 provides the effect of validating the second phase value that is based on the skin temperature measurements. Measuring the skin temperature requires skin contact between the sensor device 12 and the user's 20 skin. The user may wish to wear the sensor device with a comfortable fit that does not press the sensor device against the skin. The downside is that the skin contact may not be optimal. Furthermore, the skin contact may change during the daily activities of the user and during the night as well. Furthermore, while the fitting the skin temperature measurement data with the cosine function (also called cosinor analysis or cosinor fitting in the literature) is generally used for the circadian phase detection, the fitting may fail for several reasons. As a consequence, while the second estimate for the circadian phase improves the accuracy of the circadian phase estimation, it is advantageous to validate it before combining it with the first estimate.

In the procedure of FIG. 2, the first phase value is used for estimating the circadian phase, either alone or in combination with the second phase value. If the quality of the second phase value is determined to be high enough, e.g. the fitting is considered successful, the second phase value may be computed or the already-computed phase value may be considered eligible for the combining.

The measurement period should be at least one sleep-wake cycle i.e. 24 hours, but also a longer measurement period of several sleep-wake cycles provides better quality data. A measurement period of a week i.e. seven several sleep-wake cycles provides already very reliable data.

In an embodiment, the cosinor fitting in block 204 is performed by using least squares (LS) fitting known as such in the art. The LS fitting finds a cosine waveform (phase) that provides the lowest squared error with the measurement data. The phase of the fitted cosine waveform then represents the second phase value.

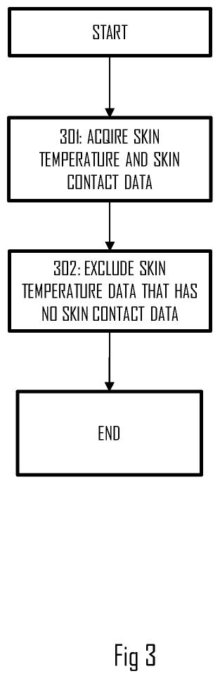

In an embodiment, the procedure of FIG. 2 comprises excluding from fitting to the cosine waveform such skin temperature measurement data for which a corresponding skin contact measurement data indicates no contact to skin. FIG. 3 is illustrating this embodiment, wherein skin contact measurement data is acquired 301 together with skin temperature measurement data for the at least one 24-hour measurement period, and such skin temperature measurements data—for which the skin contact measurement data indicating no contact to skin—is excluded 302 from the fitting to the cosine waveform in 204. As described above, a part of the skin temperature measurement data may be missing because the user 20 is not wearing the device at all for a while during the measurement period, or the device has no actual contact to user's 20 skin. The temperature measurement data associated with the lack of skin contact may indicate the ambient temperature that would degrade the performance of the fitting.

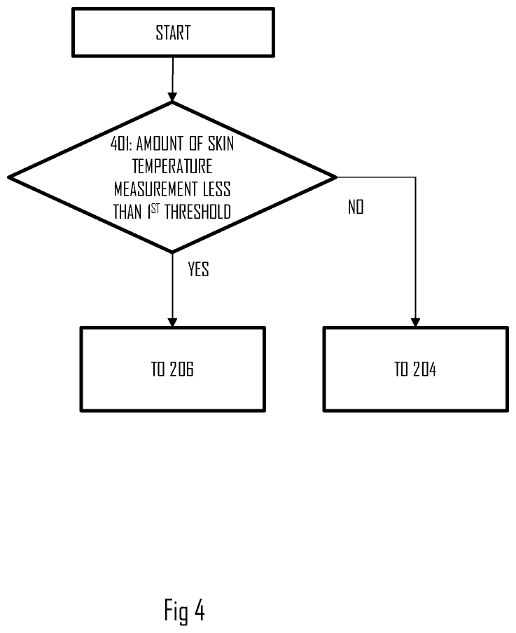

With respect to validating the skin temperature measurements and the second phase value, FIG. 4 is illustrating an embodiment of the method. Referring to FIG. 4, the procedure excluding 401 fitting of the skin temperature measurement data with the cosine waveform, if an amount of valid skin temperature measurement data associated with the skin contact measurement data indicating the skin contact is less than a first threshold during the at least one 24-hour skin temperature measurement period. In an embodiment, valid skin temperature measurement data covers at least 80% of the maximum skin temperature measurement data. This means that at least 80% of the 24-hour cycle the skin contact shall be present (at least 19.2 hours). Having less than 80% of the skin temperature measurement data may reduce the reliability of circadian rhythm detection using the skin temperature measurement. In other embodiments, a different threshold may be employed, e.g. 70%, 75%, or 85%, depending how much error the implementation tolerates and how accurate the first phase value is determined to be.

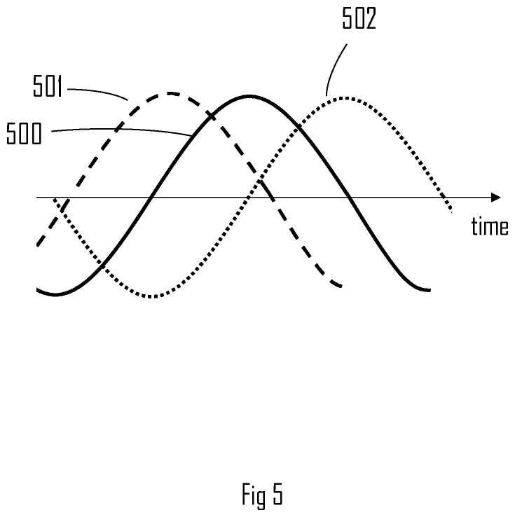

In another embodiment, the skin temperature measurements are vali-dated by evaluating the performance of the cosinor fitting of block 204. This may be carried out by arranging a phase shift between the measurement data and the cosine waveform after the fitting and evaluating a change in an error metric indicating error between the measurement data and the cosine waveform. In other words, the phase shifting is used to determine whether or not there is a better fit than that selected in 204. FIG. 5 is illustrating waveforms used in the quality evaluation of the second phase value after the fitting of the skin temperature measurement data with the cosine waveform. In this embodiment evaluating the quality of the fitting comprises the following steps
determining a first correspondence metric for the skin temperature measurement data with respect to the cosine waveform 500 after the fitting. The first correspondence metric indicates the error between the skin temperature measurement data with respect to the cosine waveform 500 after the fitting.
arranging a phase shift forward 502 and/or backward 501 between the cosine waveform 500 and the skin temperature measurement data fitted with the cosine waveform 500 and determining at least a second correspondence metric for the skin temperature measurement data with respect to the cosine waveform after the phase shift(s) 501, 502. In case multiple phase shifts are performed, multiple correspondence metrics are computed.
determining that the fit quality is above the threshold, if the second correspondence metric (and further correspondence metric(s)) indicates a greater error than the first correspondence metric, and determining that the fit quality is below the threshold, if the second correspondence metric (or at least one of the further correspondence metric(s)) indicates a smaller error than the first correspondence metric.

In other words, if the phase shift results in a fitting associated with the lower fitting error, the fitting performed in 204 has not been optimal and the circadian phase may be determined in block 206 by using only the sleek-wake rhythm. The calculation of the correspondence metrics may be done by calculating a root-mean-square-error (RMSE) of the fit i.e. the RMSE between the skin temperature measurement data and the fitted cosine waveform. Similarly, the RMSE may be calculated between the skin temperature measurement data and the shifted cosine waveforms in the case of the second correspondence metric. The RMSE should increase when the phase of the cosine waveform is varied, thus indicating that the fitting has been optimal and the phase of the fitted cosine waveform can be validated as the circadian phase estimate. The absolute value of RMSE can vary a lot between different users, but the essential thing is that the RMSE should increase when the fitted phase value is varied, forward or backward. The quality evaluation may also be carried out using a different calculation of the corresponding metrics, such as a mean-absolute-error (MAE). The correspondence metric is preferably different from the metric used for the fitting in block 204.

The result from second phase value can be ignored or its computation may be omitted, if there are too few temperature samples with verified skin contact available or if the quality evaluation analysis (e.g. RMSE based calculation) of fitted second phase value reveals that the fit was not very accurate.

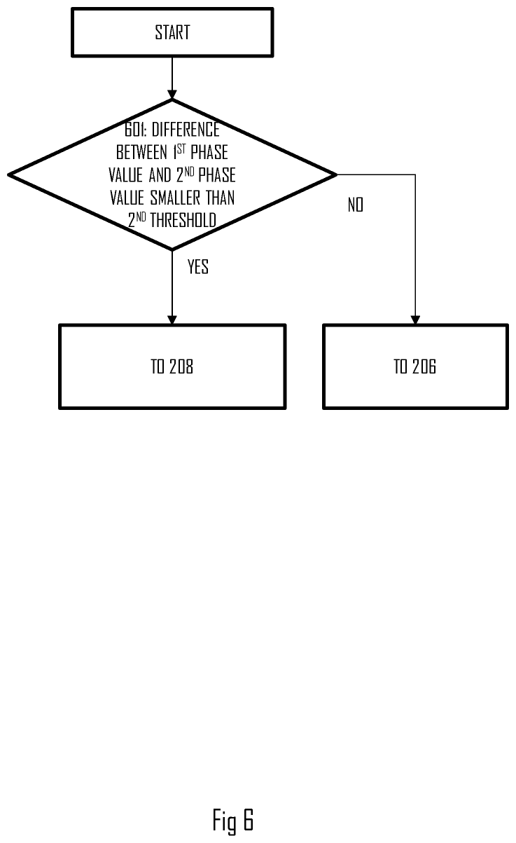

FIG. 6 is illustrating yet another embodiment for validating the second phase value, wherein the method further comprises checking 601 that if a difference between the first phase value and the second phase value is smaller than a second threshold, the circadian phase is determined 602 as a combination of the first phase value and the second phase value. The combination may be average or a weighted average of the first phase value and the second phase value. In another embodiment, the first phase value is shifted on the basis of the difference between the first phase value and the second phase value. A maximum limit for the shift may have been defined, e.g. four hours. The shifting may distinguish from the averaging 25 operation and be linear or non-linear with respect to the difference between the first phase value and the second phase value. If the difference between the first phase value and the second phase value is greater than a second threshold, the circadian phase is determined as the first phase value. The second threshold may be defined in terms of a time shift between the first and second phase value. Example values of the second threshold may be within a range from one hour to even ten hours, e.g. 1, 2, 3, 4, 5, 6, 7, 8, 9 or 10 hours. Combining this with the embodiment where the first phase value is shifted on the basis of the difference, if second threshold is 10 hours and the maximum shift is 4 hours, the first phase value may be shifted by 4 hours towards the second phase value when the difference is 10 hours. If the first 35 and second phase value are equal, no shifting of the phase value is performed. The 10 shifts when the difference is between zero and 10 hours may then be mapped in a linear or non-linear manner.

As described above, the first phase value is computed from the sleep-wake rhythm that may be based on heart activity measurement data and/or motion measurement data, while the second phase value is computed on the basis of the skin temperature measurement data fitted with the cosine waveform. While both phase values indicate the circadian phase, they are inherently in different domains because of the different measurement data and different physiological characteristics measured. Therefore, an embodiment comprises shifting one of the first phase value and the second phase value by a constant value to make the first phase value and the second phase value comparable.

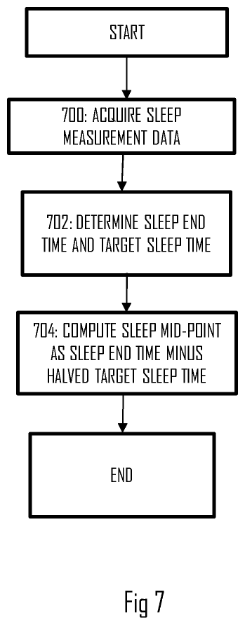

Conventional methods for estimating the circadian phase from the sleep-wake rhythm are based on determining a sleep mid-point as a mid-point between a sleep start time and sleep end time detected from the measurement data. This provides an accurate circadian phase estimate when the user's 20 sleep rhythm is regular. However, if the user reduces the sleep time by going to sleep later and waking up at the same time, the sleep mid-point gets delayed and the circadian phase is also shifted (delayed). The problem is that in such a case the user's 20 circadian phase does not actually change, but the user gains sleep debt. To overcome the problem of the user 20 changing his/her sleep times, e.g. reducing the amount of sleep but waking at the same time, an embodiment omits the sleep start time from the estimation of the first phase value. Instead, the first phase value is computed on the basis of a target sleep time (required amount of sleep which may be estimated e.g. with a sleep assistant algorithm, general sleep amount guidelines, or received as a user input), together with the sleep end time to estimate what the sleep mid-point would have been if the user 20 would have slept enough before waking up. This method provides a more accurate circadian phase estimate in a situation where the user is generating sleep debt with his/her sleeping habits. This may be realized by acquiring a sleep end time from the measurement data indicating the user's 20 sleep time and further acquiring a target sleep goal time of the user 20, determining a sleep mid-point of the user 20 on the basis of the sleep end time and the target sleep time, and computing the sleep-wake rhythm on the basis of the sleep mid-point. FIG. 7 illustrates a flow diagram of an embodiment for computing the sleep mid-point for the purpose of estimating the circadian rhythm. Referring to FIG. 7, the sleep measurement data is acquired in 700. The sleep measurement data may comprise the heart activity measurement data and/or motion measurement data that enable detection of at least the sleep end time. In 702, the sleep end time and the target sleep time are determined. The target sleep time may have been stored in the memory beforehand, and the sleep end time may be determined from the sleep measurement data acquired in 700. The sleep mid-point may be determined as a mid-point between the sleep end time and the sleep end time minus the halved target sleep time according to the following Equation in 704

sleepMidPointEstimate=sleepEndTime−TargetSleepTime/2 (1)

The purpose of computing the circadian phase may be to provide the user with information related to the circadian phase. Such information may include, for example, recommending a bedtime to the user. As known in the art, the circadian rhythm represents an internal clock and thus carries information on a natural bedtime for the user. If the user follows his/her natural rhythm, health benefits may be achieved. Another example of a parameter derivative of the circadian phase is the user's 20 chronotype. The computation of the circadian phase enables classifying a sleep chronotype of the user 20 on the basis of the circadian phase and outputting, as the parameter, the sleep chronotype via the interface. The chronotype classification may be based on observing the user's 20 circadian phase for a prolonged period of time, e.g. at least a week. The circadian phase of the night owl may be delayed with respect to the circadian phase of the early bird, for example. Yet another example of a parameter derivative of the circadian phase is an alarm of reduced alertness output to the user. For example, if the circadian phase estimation indicates that the user's 20 circadian rhythm is changing as a result of travelling across time zones or as a result of shift work, the user may be alerted that it has consequences in the form of reduced alertness. The parameters listed above may be displayed to the user via a user interface of the wearable device 11 or the user device 10, for example.

The above-described embodiments for determining the circadian phase may be used to directly infer the circadian phase or to infer a setpoint for the circadian phase. If the user lives a regular daily rhythm including a regular sleep cycle by entering sleep and waking up substantially at the same time each day, the above-described embodiments may directly indicate the user's 20 circadian phase. However, if the user changes his/her sleep-wake rhythm as a result of travelling over time zones, shift work etc., it takes time for the circadian phase to adapt to the new sleep-wake rhythm. In such a case, the above-described embodiments may define a setpoint for the circadian phase towards which the circadian phase adopts at a certain daily adaptation rate. In such a case, the circadian phase for today may be determined on the basis of the setpoint, the adaptation rate, and the circadian phase yesterday. Below, embodiments for determining the adaptation rate are described.

The above-described embodiments for estimating the circadian phase may be utilized for the purpose of detecting the user's 20 alertness, providing guidance related to the user's 20 health, fitness, or training, or it may also be used for detecting changes in the circadian phase. As described in Background, the changes in the circadian phase may be caused by various reasons affecting the user's 20 sleep-wake rhythm, and it may be beneficial to estimate and output to the user information on how long it takes for the user's 20 body to settle to the changed sleep-wake rhythm. However, it should be appreciated that the circadian phase may be determined in the embodiments below by using other methods, e.g. one according to state of the art.

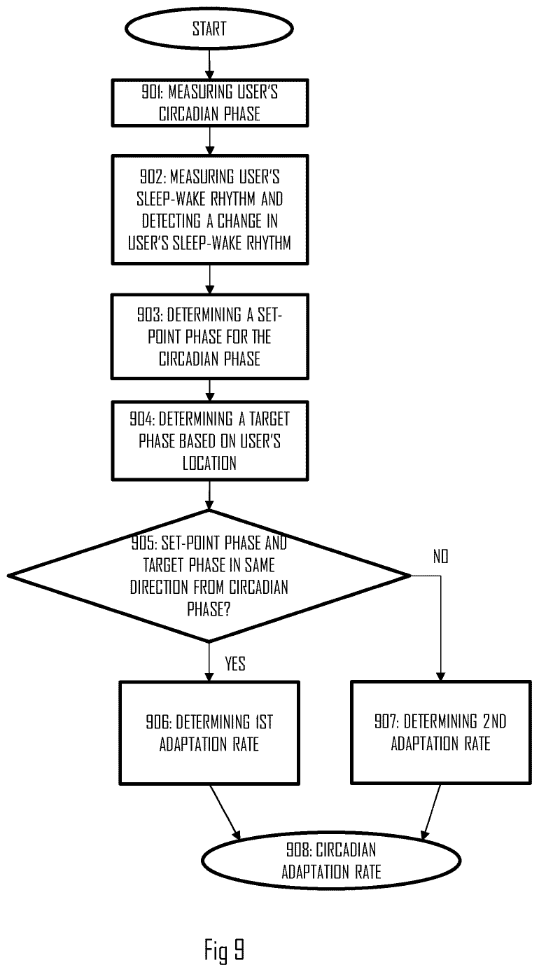

FIG. 9 is illustrating an embodiment of a method for estimating adaptation of a circadian rhythm of a user, wherein the method comprises measuring 901 the user's 20 circadian phase by using at least one of a skin temperature sensor, a heart activity sensor, and a motion sensor, measuring 902 the user's 20 sleep-wake rhythm by using at least one of the heart activity sensor and the motion sensor, and detecting a change in the user's 20 sleep-wake rhythm on the basis of the measurements, determining 903 a setpoint phase for the circadian phase on the basis of the changed sleep-wake rhythm, determining 904 a target circadian phase based on the user's 20 location, determining 906 a circadian adaptation rate based on a first adaptation rate, if the setpoint phase and the target phase are in the same direction with respect to the current circadian phase, and determining 907 the circadian adaptation rate based on a second adaptation rate, if the setpoint phase and the target phase are to opposite directions from the current circadian phase, wherein the second adaptation rate is smaller than the first adaptation rate, and outputting 908 the determined circadian adaptation rate or a parameter derivative of the determined circadian adaptation rate via an interface to be presented to the user.

Using the target circadian phase based on the user's 20 location as a reference point for the adaptation rate of the circadian phase provides a more accurate estimate of the adaptation rate than the conventional solution, particularly when the user is changing his/her sleep-wake rhythm away from the target circadian phase.

In an embodiment, the adaptation rate is indicated to the user visually via a user interface of the wearable device (e.g. the wrist device) or the user device. The visual output may additionally comprise an indicator of the current circadian phase with respect to the setpoint. Additionally, the target circadian phase may be displayed to the user. The parameter derivative of the computed circadian adaptation rate is an indicator indicating the distance between the current circadian phase and the setpoint phase. The (daily) evolvement of such an indicator is also an indicator of the adaptation rate. Yet another parameter derivative of the adaptation rate is a smart instruction to the user. For example, if the adaptation rate is non-zero, the apparatus performing the procedure of FIG. 9 may output to the user an indication that the circadian phase is currently adapting and adapt a training schedule of the user such that the intensity of the training schedule is reduced for at least a part of the duration when the circadian phase is adapting. As a consequence, the apparatus may modify at least one exercise plan of the training schedule by reducing the intensity of at least one exercise. Yet another example of the parameter is a sleep time proposed to the user. As the circadian phase is shifting, so is the optimal bedtime for the user, and the proposed sleep time is a function of the determined adaptation rate. The new sleep time may be adapted to the same direction as where the circadian phase is adapting and by the amount of the (daily) adaptation rate, for example.

In an embodiment, also the temperature sensor is used for determining the circadian phase. In an embodiment, the circadian phase is determined by using the same sensors used for determining the sleep-wake rhythm, e.g. any combination of the heart activity sensor, motion sensor, and the skin temperature sensor.

The circadian rhythm adapts to changes in the sleep-wake rhythm. For example, if there is a fast and drastic change in the sleep timing (e.g. time zone change), the circadian rhythm starts to gradually shift towards the new, changed sleep timing instead of jumping to follow the new sleep rhythm immediately. The adaptation rate is a function of the changed sleep-wake rhythm and, additionally, a function of a natural circadian rhythm for the (possibly new) geographical location of the user. Different geographical locations on Earth have different sunlight rhythms and, as described in Background, the sunlight rhythm affects the natural circadian rhythm for the particular location. Furthermore, the sunlight rhythm at the location of the user affects the adaptation rate, and the embodiments described herein quantize this characteristic, thereby providing a more accurate estimate of the circadian phase adaptation rate.

Furthermore, the method uses wrist skin temperature and sleep wake-up times to determine circadian rhythm characteristics. The 24-h profile of wrist skin temperature provides a feasible measure of the circadian phase in free-living environments. It correlates with the secretion pattern of melatonin, also known as “sleep hormone”. The sleep-wake rhythm may be computed from the heart activity measurement data and/or activity measurement data. There exist several commercially available solutions and products for detecting the sleep start time and sleep end time on the basis of such measurement data, so detailed description thereof is omitted.

In some embodiments, e.g. in the absence of wrist skin temperature data, or as a supplement to the wrist skin temperature data, the circadian rhythm characteristics may be derived from the measurement data used for determining the sleep-wake rhythm, as described above in connection with FIG. 2, for example. For the purpose of estimating the circadian phase on the basis of the sleep-wake rhythm alone, previous values of the circadian phase may also be utilized. As described above, the circadian phase does not immediately adapt to the changed sleep-wake rhythm. Other methods can be used to estimate the circadian rhythm characteristics (particularly the circadian phase).

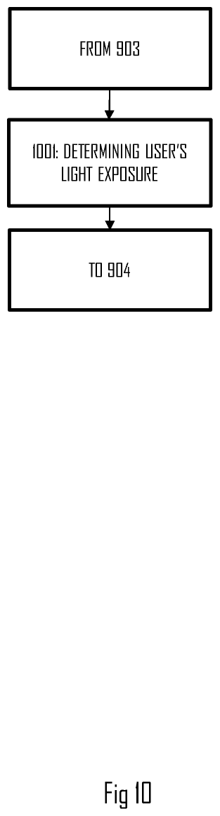

In an embodiment, the target circadian phase is determined independently of the user's 20 measured sleep-wake rhythm. As described above, the target circadian phase is linked to the location of the user while the sleep-wake rhythm is determined on the basis of the heart activity and/or motion measurement data measured on the user. FIG. 10 is illustrating such an embodiment where the target phase is determined 1001 by user's 20 light exposure at user's 20 current location. The light exposure may be exposure to sun or artificial light. In case of the sun light, the adaptation rate is estimated utilizing the information of a local sun-light rhythm at the user's 20 location. In particular, the target circadian phase is determined by utilizing the information of the local sun-light rhythm and, since the adaptation direction with respect to the target circadian phase affects the adaptation rate, the target circadian phase is used to determine the adaptation rate. The local sun-light rhythm may be defined using e.g. global navigation satellite system (GNSS) based location information of the user or by current time-zone information. Current time-zone information may be retrieved e.g. from wrist device 11 itself or from a serving telecom network operator or from an internet service.

In another embodiment, the user's 20 light exposure is estimated by the user's 20 location in longitude and latitude coordinates, wherein the light exposure is different for different latitude coordinates associated with the same longitude coordinates. Using the latitude coordinates in addition to the longitude coordinates improves the accuracy of the target circadian phase so that it matches better with the local sunlight rhythm. The light exposure estimation may also take into account the time of year together with the longitude and latitude coordinates as the light exposure depends also on the time of the year. Again, the coordinates may be retrieved using e.g. global navigation satellite system (GNSS) based location solution of the wrist device 11 or from a serving telecom network operator or from an internet service.

In another embodiment the light exposure is determined on the basis of time of year. The method may take into account when there is a transition into and out of daylight-saving time. The time of the year information may be retrieved from the wrist device's 11 internal clock or from a serving telecom network operator or from an internet service.

In another embodiment the light exposure is determined with a light sensor.

In another embodiment the user's 20 light exposure is estimated primarily on the basis of the measured light exposure and, if the measured light exposure is not available, on the basis of the user's 20 location in longitude and latitude coordinates. Thus, the measured light exposure may be prioritized over the geographical location.

In the embodiments based on the sunlight rhythm at the user's 20 location, the apparatus performing the procedure of FIG. 2 may store definitions as how to convert the location and/or light exposure information to the target circadian phase. The definitions may follow the logic that the user should sleep when it is dark and be awake during the greatest light exposure, following the logic of natural circadian rhythm. Depending on the light exposure at the location, the exact bedtime and wake-up time may however vary.

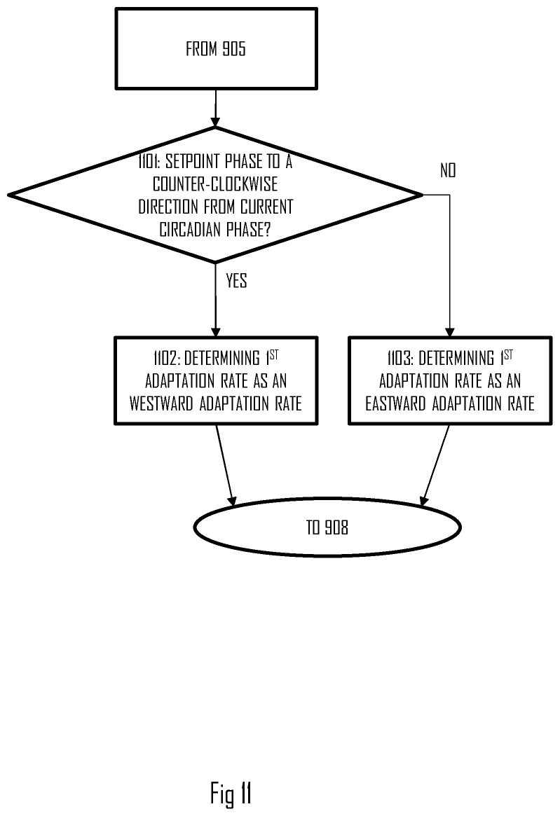

FIG. 11 is illustrating another embodiment, wherein the first adaptation rate is determined 1102 as a westward adaptation rate if the setpoint phase is to a clockwise direction with respect to the current circadian phase, and the first adaptation rate is determined 1103 as an eastward adaptation rate if the setpoint phase is to a counter-clockwise direction with respect to the current circadian phase. The clockwise direction may be understood such that the setpoint phase is decayed (becomes later) with respect to the current circadian phase (the circadian phase is being decayed towards the setpoint phase), and the counter-clockwise direction refers to the current circadian phase being advanced (becomes earlier) with respect to the setpoint phase (the circadian phase is being advanced towards the setpoint phase). In 1101 the direction of the first adaptation rate is checked. The user's 20 adaptation is faster in a “westward” change (a change from an “earlier” time-zone to a “later” time-zone, e.g. from UTC to UTC-4) compared to an eastward change. The westward adaptation rate is preferably greater than the eastward adaptation rate. In an embodiment, the westward adaptation rate (clockwise direction) is 1 hour per day or about one hour per day, while the eastward adaptation rate (counter-clockwise direction) is less than one hour per day, e.g. 0.5 to 0.7 hours per day. The adaptation rate away from the target circadian phase may be smaller than the eastward adaptation rate, e.g. smaller than 0.5 hours per day such as between 0.3 and 0.4 hours per day.

If the circadian phase adapts away from the target circadian phase, the adaptation rate may be the same for the eastward direction as for the westward direction.

In another embodiment, the method including the measurements is performed in a wrist computer or a wrist device.

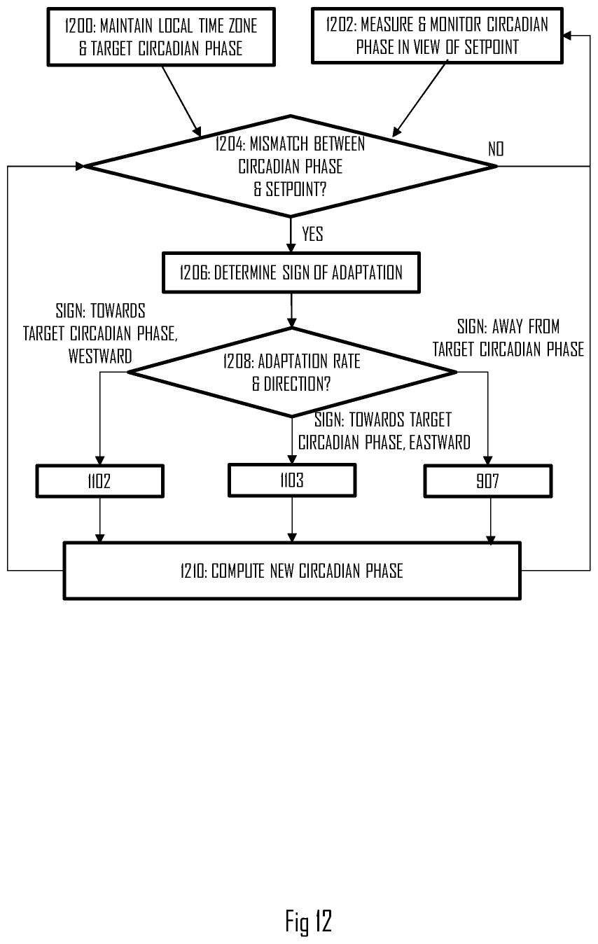

The above-described procedures of determining the circadian phase and the adaptation rate may be carried out as a combined iterative procedure performed repeatedly, e.g. daily. FIG. 12 illustrates an embodiment of such a procedure. As described above, the setpoint phase may be computed on the basis of the user's 20 sleep-wake rhythm. In case the sleep-wake rhythm has remained unchanged for a certain period of time so that the circadian rhythm is aligned with the sleep-wake rhythm, the setpoint phase equals to the circadian phase. A change in the sleep-wake rhythm triggers the change to the circadian phase.

Referring to FIG. 12, the apparatus (e.g. the wearable device or the user device) may in 1200 maintain the target circadian phase for the user's 20 current location, e.g. for the local time zone. In 1202, the apparatus may maintain information on the circadian phase and, further, the setpoint phase that is based on the sleep-wake rhythm. Block 1204 evaluates mismatch (a difference) between the circadian phase and the setpoint phase. The threshold may be determined on the basis of the adaptation rates, e.g. to be between 0.3 and 1 hours. If the difference is below a determined threshold, the process may return to 1202. If the difference is above the determined threshold, the adaptation of the circadian phase may be triggered, and the process may proceed to 1206. In 1206, a sign of the adaptation direction is determined on the basis of the target circadian phase. The sign indicates whether the adaptation of the circadian phase is towards the target circadian phase or away from it. In 1208, the adaptation rate is determined on the basis of the direction of the adaptation towards the setpoint phase (clockwise or counter-clock-wise) and the sign determined in 1206. If the sign indicates that the adaptation is away from the target circadian phase, the process may proceed to 907 where the slowest adaptation rate is selected. If the sign indicates that the adaptation is towards the target circadian phase in the counter-clockwise direction, the fastest adaptation rate may be selected (1102). If the sign indicates that the adaptation is towards the target circadian phase in the clockwise direction, the second fastest (or second slowest) adaptation rate of these three adaptation rates may be selected (1103). After selecting the adaptation rate, the new circadian phase may be computed 1210 on the basis of the circadian phase used in 1204 for detecting the mismatch and the selected adaptation rate. Thereafter, the process may return to 1202 to wait for new measurement data and new value of the setpoint phase.

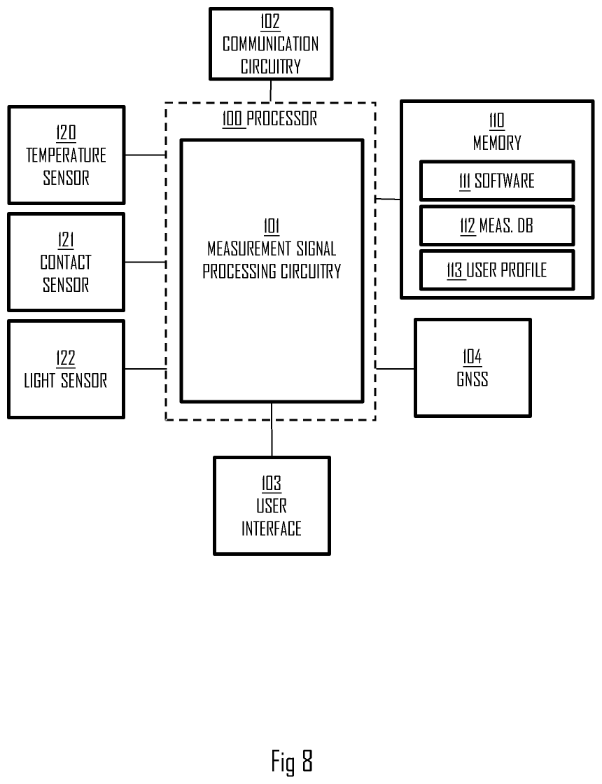

FIG. 8 illustrates an embodiment of an apparatus configured to carry out at least some of the above-described functions in detecting a user's 20 circadian rhythm. The apparatus may comprise an electronic device comprising at least one processor 100 and at least one memory 110. The processor 100 may form or be a part of a processing circuitry. The apparatus may further comprise a user interface 103 comprising a display screen or another display unit, an input device such as one or more buttons and/or a touch-sensitive surface, and an audio output device such as a loudspeaker.

The processor 100 may comprise a measurement signal processing circuitry 101 configured to detect the user's 20 circadian rhythm by carrying out the procedure of FIG. 9 or any one of the embodiments thereof described above. In an embodiment, the measurement signal processing circuitry is further configured to carry out the procedure of FIG. 2 or any one of the embodiments thereof described above.

The apparatus may comprise a communication circuitry 102 connected to the processor 100. The processor 100 may use the communication circuitry 102 to transmit and receive frames according to the supported wireless communication protocol. In some embodiments, the processor 100 may use the communication circuitry 102 to transmit the data on the user's 20 circadian rhythm, sleep data and/or other parameters to another apparatus, e.g. to a cloud server storing the user's 20 user account.

In an embodiment, the apparatus comprises at least one skin temperature sensor 120. Additionally, the apparatus may communicate with at least one skin contact sensor 121. The skin contact sensor 121 may use optical or bioimpedance method. In an embodiment, the apparatus comprises a light sensor 122 to measure the light exposure and further determine the sun-light rhythm of the user's 20 location.

In the embodiments, the devices may comprise a global positioning system or circuitry 104 such as the Global Positioning System or another satellite navigation system to provide location information to be used in determining the user's 20 location. The apparatus may also receive the location information from the internet using the communication circuitry 102.

The memory 110 may store a computer program product 111 circadian rhythm detection algorithm the processor executes upon reading the computer program. The memory may further store a user profile 113 of the user 20 storing personal characteristics of the user 20. The memory may further store a measurement database 112 comprising the measured history of sleep-wake rhythm, circadian rhythm, and user's 20 preference or plans related to planned events such as travelling over time zones, daylight saving time transitions or shift work schedules.

As used in this application, the term ‘circuitry’ refers to all of the following: (a) hardware-only circuit implementations, such as implementations in only analog and/or digital circuitry, and (b) combinations of circuits and software (and/or firmware), such as (as applicable): (i) a combination of processor(s) or (ii) portions of processor(s)/software including digital signal processor(s), software, and memory(ies) that work together to cause an apparatus to perform various functions, and (c) circuits, such as a microprocessor(s) or a portion of a microprocessor(s), that require software or firmware for operation, even if the software or firmware is not physically present. This definition of ‘circuitry’ applies to all uses of this term in this application. As a further example, as used in this application, the term ‘circuitry’ would also cover an implementation of merely a processor (or multiple processors) or a portion of a processor and its (or their) accompanying software and/or firmware. The term ‘circuitry’ would also cover, for example and if applicable to the particular element, a baseband integrated circuit or applications processor integrated circuit for a mobile phone or a similar integrated circuit in a server, a cellular network device, or another network device.

In an embodiment, at least some of the processes described in connection with FIGS. 2-4, 6-7 and 9-12 may be carried out by an apparatus comprising corresponding means for carrying out at least some of the described processes. Some example means for carrying out the processes may include at least one of the following: detector, processor (including dual-core and multiple-core processors), digital signal processor, controller, receiver, transmitter, encoder, decoder, memory, RAM, ROM, software, firmware, display, user interface, display circuitry, user interface circuitry, user interface software, display software, circuit, and circuitry. In an embodiment, the at least one processor 100, the memory 110, and the computer program code 118 form processing means or comprises one or more computer program code portions for carrying out one or more operations according to any one of the embodiments of FIGS. 2-4, 6-7 and 9-12 or operations thereof.

The techniques and methods described herein may be implemented by various means. For example, these techniques may be implemented in hardware (one or more devices), firmware (one or more devices), software (one or more modules), or combinations thereof. For a hardware implementation, the apparatus(es) of embodiments may be implemented within one or more application-specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. For firmware or software, the implementation can be carried out through modules of at least one chipset (e.g. procedures, functions, and so on) that perform the functions described herein. The software codes may be stored in a memory unit and executed by processors. The memory unit may be implemented within the processor or externally to the processor. In the latter case, it can be communicatively coupled to the processor via various means, as is known in the art. Additionally, the components of the systems described herein may be rear-ranged and/or complemented by additional components in order to facilitate the achievements of the various aspects, etc., described with regard thereto, and they are not limited to the precise configurations set forth in the given figures, as will be appreciated by one skilled in the art.

Embodiments as described may also be carried out in the form of a computer process defined by a computer program or portions thereof. Embodiments of the methods described in connection with FIGS. 2-4, 6-7 and 9-12 may be carried out by executing at least one portion of a computer program comprising corresponding instructions. The computer program may be in source code form, object code form, or in some intermediate form, and it may be stored in some sort of carrier, which may be any entity or device capable of carrying the program. For example, the computer program may be stored on a computer program distribution medium readable by a computer or a processor. The computer program medium may be, for example but not limited to, a record medium, computer memory, read-only memory, electrical carrier signal, telecommunications signal, and software distribution package, for example. The computer program medium may be a non-transitory medium. Coding of software for carrying out the embodiments as shown and described is well within the scope of a person of ordinary skill in the art.

It will be obvious to a person skilled in the art that, as the technology advances, the inventive concept can be implemented in various ways. The invention and its embodiments are not limited to the examples described above but may vary within the scope of the claims.

What is claimed is:

1. A method for estimating an adaptation rate of a circadian rhythm of a user, the method comprising:
measuring the user's circadian phase by using at least one of a skin temperature sensor, a heart activity sensor, and a motion sensor;
measuring the user's sleep-wake rhythm by using at least one of said heart activity sensor and the motion sensor, and detecting a change in the user's sleep-wake rhythm on the basis of the measurements;
determining a setpoint phase for the circadian phase for the changed sleep-wake rhythm towards which the changed sleep-wake rhythm adopts at a daily adaptation rate;
determining a target circadian phase based on the user's location;
determining said circadian adaptation rate indicative of rate that the user's change sleep-wake rhythm is temporally progressing towards/away from the setpoint phase;
determining a parameter derivative of the computed circadian adaptation rate indicative of the temporal distance between a current circadian phase and the setpoint phase and whether said rate is progressing toward the target circadian phase or away from the target circadian phase, and where the circadian adaptation rate is:
based on a first adaptation rate, if current circadian phase is progressing toward the setpoint phase and the target phase, and
based on a second adaptation rate, if current circadian phase is progressing away the setpoint phase and the target phase, wherein the second adaptation rate is smaller than the first adaptation rate; and
outputting the determined circadian adaptation rate or a parameter derivative of the computed circadian adaptation rate via an interface to be presented to the user.

2. The method according to claim 1, wherein the target circadian phase is determined independently of the user's measured sleep-wake rhythm.

3. The method according to claim 2, wherein the target phase is determined by user's light exposure at user's current location.

4. The method according to claim 3, wherein the user's light exposure is estimated by the user's location in longitude and latitude coordinates, wherein the light exposure is different for different latitude coordinates associated with the same longitude coordinates.

5. The method according to claim 4, wherein the light exposure is further determined on the basis of time of year.

6. The method according to claim 3, further comprising measuring the user's light exposure with a light sensor.

7. The method according to claim 6, wherein the user's light exposure is estimated on the basis of the measured light exposure and, if the measured light exposure is not available, on the basis of the user's location in longitude and latitude coordinates.

8. The method according to claim 1, wherein, the first adaptation rate is a westward adaptation rate if the setpoint phase is to a counter-clockwise direction with respect to the current circadian phase; and the first adaptation rate is an eastward adaptation rate if the setpoint phase is to a clockwise direction with respect to the current circadian phase, wherein the eastward adaptation rate is smaller than the westward adaptation rate.

9. The method according to claim 1, further comprising performing the measurements in a wrist computer.

10. An apparatus for estimating an adaptation rate of a circadian rhythm of a user, the apparatus performing operations comprising:
measuring the user's circadian phase by using at least one of a skin temperature sensor, a heart activity sensor, and a motion sensor;
measuring the user's sleep-wake rhythm by using at least one of the heart activity sensor and the motion sensor, and detecting a change in the user's sleep-wake rhythm on the basis of the measurements;
determining a setpoint phase for the circadian phase for the changed sleep-wake rhythm towards which the changed sleep-wake rhythm adopts at a daily adaptation rate;
determining a target circadian phase based on the user's location;
determining a circadian adaptation rate indicative of rate that the user's change sleep-wake rhythm is temporally progressing towards/away from the setpoint phase;
determining a parameter derivative of the computed circadian adaptation rate indicative of the temporal distance between a current circadian phase and the setpoint phase and determining whether said rate is progressing toward the target circadian phase or away from the target circadian phase where the circadian adaptation rate is:
based on a first adaptation rate, if current circadian phase is progressing toward the setpoint phase and the target phase, and
based on a second adaptation rate, if current circadian phase is progressing away the setpoint phase and the target phase, wherein the second adaptation rate is smaller than the first adaptation rate; and
outputting the determined circadian adaptation rate or a parameter derivative of the computed circadian adaptation rate via an interface to be presented to the user.

11. The apparatus according to claim 10, further comprising means to determine the target circadian phase independently of the user's measured sleep-wake rhythm.

12. The apparatus according to claim 10, further comprising an attachment mechanism configured to attach the apparatus to a wrist.

13. A computer program product embodied on a distribution medium readable by a computer and comprising instructions that when executed by an apparatus, perform operations comprising:
measuring the user's circadian phase by using at least one of a skin temperature sensor, a heart activity sensor, and a motion sensor;
measuring the user's sleep-wake rhythm by using at least one of the heart activity sensor and the motion sensor, and detecting a change in the user's sleep-wake rhythm on the basis of the measurements;
determining a setpoint phase for the circadian phase for the changed sleep-wake rhythm towards which the changed sleep-wake rhythm adopts at a daily adaptation rate;
determining a target circadian phase based on the user's location;
determining a circadian adaptation rate indicative of rate that the user's change sleep-wake rhythm is temporally progressing towards/away from the setpoint phase;
determining a parameter derivative of the computed circadian adaptation rate indicative of the temporal distance between a current circadian phase and the setpoint phase and whether said rate is progressing toward the target circadian phase or away from the target circadian phase, and where the circadian adaptation rate is:
based on a first adaptation rate, if current circadian phase is progressing toward the setpoint phase and the target phase, and
based on a second adaptation rate, if current circadian phase is progressing away the setpoint phase and the target phase, wherein the second adaptation rate is smaller than the first adaptation rate; and
outputting the determined circadian adaptation rate or a parameter derivative of the determined circadian adaptation rate via an interface to be presented to the user.

14. The computer program product according to claim 13 embodied on a distribution medium readable by a computer and comprising instructions, that when executed by an apparatus, perform operations comprising determining the target circadian phase independently of the user's measured sleep-wake rhythm.
